SEQUENCE LISTING (4 sequences)

SEQ ID NO: 1 - protein, 473 aa, Thermotoga maritima
MMYPMPSWVYDSVVYQIFPDRFFIGKGKTVEDKKDLYLKRGGVIEKWGVPPRKLPGAQHVKIFYGGDLWGIAEKVDYFEELGINVLYLTPIFLSDTNHKYDTIDYFRVDPQFGGKRAFLHLLRVLHERSMKLILDGVFNHVGSQHPWFKKAKKNDPEYVNRFFLYKDRHRSWFDVGSLPELNVEVEEVKEYILKVVEHYLKLGIDGWRLDCGHDLGPTVNLWINMKVKEFSAEKYLVSEIWTYPAGWDMVDGLMNYNFRNLVLSYVNGETDSIGFHLERAYRETKNIFGCWNMLDSHDTPRLATMVPDRDLRKLAVVLQFTYPGVPLVYYGTEIGLTGGEDPECRATMEWNREKWDVDLFEFYKKMIRLRRTDPGLRFGEFVLLNDSPLAFLRKAPHPLQNTIVVVNPGEEKVLVLSIPDGKIMNTTPLVDVFSGERFHVDGGVVKLPLLARSFRILKPEDLRVGKYRLYKRV

SEQ ID NO: 2 - protein, 472 aa, Thermotoga neapolitana
MYPIPSWVYDSVVYQIFPDRFFIGKGKTVEDKKDLYLKRGGTIEKWGVPPRKLPGAQHVKVFYGGDLWGIAEKIDYLEELGVNAVYLTPIFLSDTNHKYDTIDYFKIDPQFGGKRAFVHLLKVLHSRNIKLILDGVFNHVGSQHPWFKKARKKDPEYVNRFFLYRDRHRSWFDVGSLPELNVEVEEVREYILKVVQHYLEVGVDGWRLDCGHDLGPLVNLWINMKVKEFSSEKYLVSEIWTYPAGWEMVDGLMNYNFRSLVLSYVNGETDSIGTELERAYRETKNIFGCWNMLDSHDTPRLATTVPVKDLRKLAIVLQFTYPGVPLVYYGTEIGLTGGEDPECRATMEWNREKWDMELFEFYKKMIRFRRTDPGLRFGEFILLKEKPLAFMRKAPHPLQDTIVVVNPEEEKNVVLSLPDGKIMNATPLFDIFTGEKFHVDGGVVKVPVGRRSFRILKPIDLRVGRYRLYKRV

SEQ ID NO: 3 - DNA, 37 bp, Artificial
  oth88
gttcaacatatgtatcccataccaagttgggtatacg

SEQ ID NO: 4 - DNA, 43 bp, Artificial
  oth89
atgtatgcggccgctcacactctcttgtacaatctgtaccttc

(19) United States
(12) Patent Application Publication
(10) Pub. No.: US 2008/0124427 A1
(43) Pub. Date: May 29, 2008

(54) Process for preparing a dough comprising a starch-degrading glucogenic exo-amy-lase of family 13

(75) Inventors: Thomas Schäfe, Farum (DK); Tina Spendler, Malev (DK); Tina Hoff, Holte (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/569,242

(22) PCT Filed: Aug. 23, 2004
(86) PCT No.: PCT/DK04/00559

§ 371 Date: Feb. 22, 2006

Related U.S. Application Data
(60) Provisional application No. 60/497,411, filed on Aug. 22, 2003.

(30) Foreign Application Priority Data
Aug. 22, 2003 (DK) PA 2003 01202

(51) Int. Cl. A21D 8/02; C07H 21/04; C12P 21/06; C12N 9/32; C12N 1/18; C12N 15/74

(52) U.S. Cl. 426/20; 435/204; 435/69.1; 435/254.2; 435/483; 536/23.2

(57) ABSTRACT

The staling of an edible product made from dough can be retarded by adding a starch-degrading glucogenic exo-amylase of Family 13 to the dough, particularly an amylase from Thermotoga.

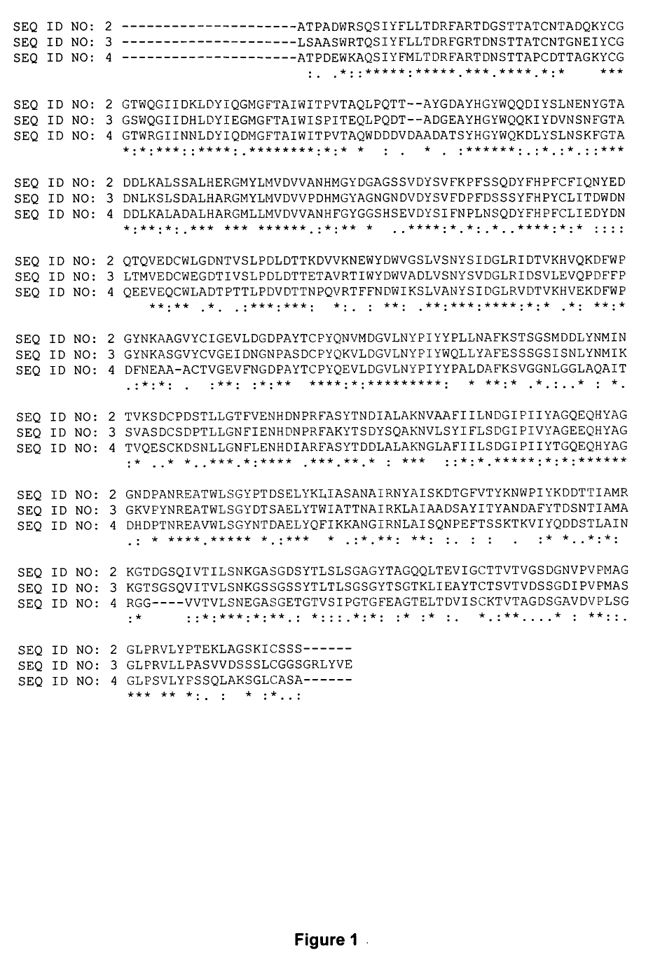

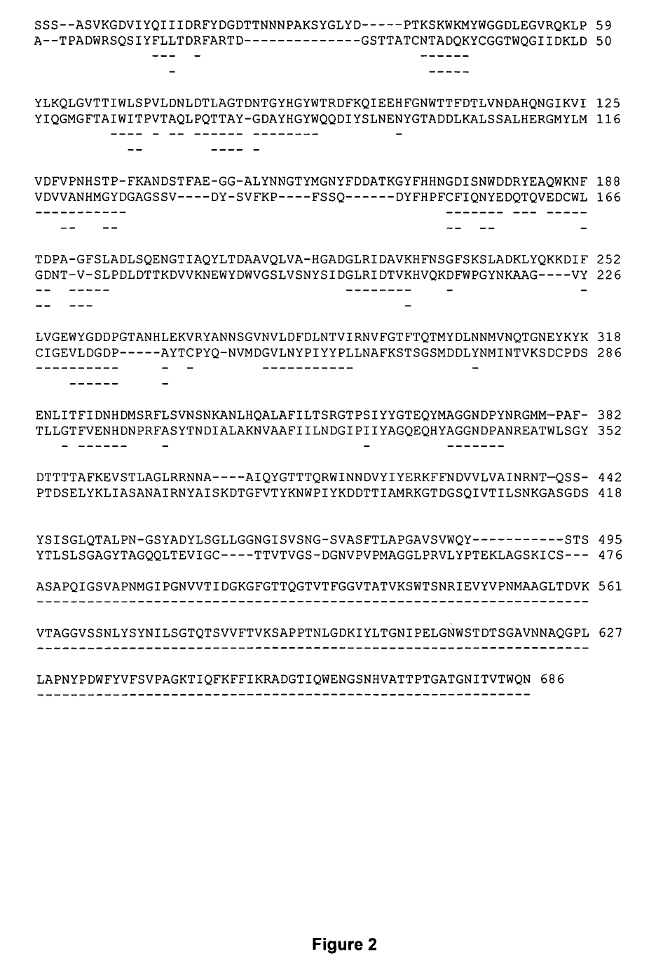

### FIELD OF THE INVENTION

The present invention relates to a process for preparing a dough or an edible product made from dough, e.g. by baking or steaming. More particularly, it relates to such a process where the edible product has retarded staling.

### BACKGROUND OF THE INVENTION

EP 494233 discloses the addition to dough of a maltogenic exo-amylase in order to retard the staling of a baked product made from the dough. The maltogenic exo-amylase is further described in EP 120693.

The following describe the addition of various enzymes to dough: DE 19855352, EP 412607, WO 9950399, U.S. Pat. No. 6,579,546, U.S. Pat. No. 4,160,848, EP 686348, US 2002028267.

M-H Lee et al., Biochemical and Biophysical Research Communications, 295 (2002), 818-825 describes an amylolytic enzyme from Thermotoga maritima.

### SUMMARY OF THE INVENTION

The inventors have found that the staling of an edible product made by leavening and heating a dough can be retarded by adding a starch-degrading glucogenic exo-amylase of Family 13 to the dough.

Accordingly, the invention provides a process for preparing a dough or an edible product made from dough, which process comprises adding a starch-degrading glucogenic exo-amylase of Family 13 to the dough. The invention also provides a composition for use in this process.

### DETAILED DESCRIPTION OF THE INVENTION

### Starch-Degrading Glucogenic Exo-Amylase of Family 13

The invention uses an enzyme which has the ability to degrade starch or amylopectin by releasing glucose as the major product. It may release glucose from the reducing end. The starch-degrading glucogenic exo-amylase of Family 13 may also have the ability to hydrolyze maltooligosaccharides, e.g. with 3-7 glucose units.

The exo-amylase used in the invention belongs to Family 13 according to the classification based on amino acid sequence similarities, as described, e.g., in the following literature:
Henrissat B., A classification of glycosyl hydrolases based on amino-acid sequence similarities. Biochem. J. 280:309-316(1991).
Henrissat B., Bairoch A. New families in the classification of glycosyl hydrolases based on amino-acid sequence similarities. Biochem. J. 293:781-788(1993).
Henrissat B., Bairoch A. Updating the sequence-based classification of glycosyl hydrolases. Biochem. J. 316:695-696(1996).
Davies G., Henrissat B. Structures and mechanisms of glycosyl hydrolases. Structure 3:853-859(1995).

The starch-degrading glucogenic exo-amylase of Family 13 may be obtained from a microbial source, such as bacteria, e.g. Thermotoga, particularly T. maritima or T. neapolitana, more particularly the strain MSB8. Some particular examples of exo-amylases are:
An exo-amylase from T. maritima described by M-H Lee et al., Biochem. Biophys. Res. Comm. 295 (2002) 818-825. It has optimum temperature and pH at 85° C. and 6.5. It retains 80% of the activity at 90° C., but the residual activity is greatly reduced at 95° C.
An exo-amylase from T. neapolitana, prepared e.g. as described in the examples from the strain DSM 4359 (commercially available from DSMZ-Deutsche Sammlung von Mikroorganismen und Zellkulturen GmbH, Mascheroder Weg 1b, Braunschweig, Germany)
Exo-amylases from T. maritima and T. neapolitana having the amino acid sequences shown in SEQ ID NO: 1 and 2, the two sequences having about 89% amino acid identity.
An exo-amylase having at least 80% identity to SEQ ID NO: 1 or 2, particularly at least 85%, at least 90% or at least 95% identity.

The starch-degrading glucogenic exo-amylase of Family 13 may be chosen so as to have optimum pH of 4-7 and optimum temperature of 70-100° C., particularly 80-90° C. The exo-amylase may be used at a dosage of 1-15 mg enzyme protein per kg flour, particularly 2-10 mg/kg.

### Dough

The dough may be leavened e.g. by adding chemical leavening agents or yeast, usually Saccharomyces cerevisiae (baker's yeast).

The dough generally comprises meal, flour or starch such as wheat meal, wheat flour, corn flour, corn starch, rye meal, rye flour, oat flour, oat meal, sorghum meal, sorghum flour, rice flour, potato meal, potato flour or potato starch.

The dough may be fresh, frozen or par-baked.

The dough may be a laminated dough.

The dough may also comprise other conventional dough ingredients, e.g.: proteins, such as milk powder and gluten; eggs (either whole eggs, egg yolks or egg whites); an oxidant such as ascorbic acid, potassium bromate, potassium iodate, azodicarbonamide (ADA) or ammonium persulfate; an amino acid such as L-cysteine; a sugar; a salt such as sodium chloride, calcium acetate, sodium sulfate or calcium sulfate. The dough may comprise fat (triglyceride) such as granulated fat or shortening.

The dough may further comprise an emulsifier such as mono- or diglycerides, diacetyl tartaric acid esters of mono- or diglycerides, sugar esters of fatty acids, polyglycerol esters of fatty acids, lactic acid esters of monoglycerides, acetic acid esters of monoglycerides, poly-oxyethylene stearates, or lysolecithin.

### Edible Product

The dough may be used to prepare an edible product, e.g. by leavening the dough and heating it, e.g. by baking or steaming. The product may be of a soft or a crisp character, either of a white, light or dark type. Examples are steamed or baked bread (in particular white, whole-meal or rye bread), typically in the form of loaves or rolls, French baguette-type bread, pita bread, tortillas, cakes, pancakes, biscuits, cookies, pie crusts, crisp bread, steamed bread, pizza and the like.

### Optional Additional Enzyme

The starch-degrading glucogenic exo-amylase of Family 13 may optionally be used together with one or more additional enzymes.

The additional enzyme may be a lipolytic enzyme, particularly phospholipase, galactoilipase and/or triacyl glycerol lipase activity, e.g. as described in WO 9953769, WO 0032758, WO 0200852 or WO 2002066622.

Further, the additional enzyme may be a second amylase, a cyclodextrin glucanotransferase, a protease or peptidase, in particular an exopeptidase, a transglutaminase, a lipase, a phospholipase, a cellulase, a hemicellulase, a glycosyltransferase, a branching enzyme (1,4-α-glucan branching enzyme) or an oxidoreductase. The additional enzyme may be of mammalian, plant or microbial (bacterial, yeast or fungal) origin.

The second amylase may be from a fungus, bacterium or plant. It may be a maltogenic alpha-amylase (EC 3.2.1.133), e.g. from B. stearothermophilus, an alpha-amylase, e.g. from Bacillus, particularly B. licheniformis or B. amyloliquefaciens, a beta-amylase, e.g. from plant (e.g. soy bean) or from microbial sources (e.g. Bacillus), a glucoamylase, e.g. from A. niger, or a fungal alpha-amylase, e.g. from A. oryzae.

The hemicellulase may be a pentosanase, e.g. a xylanase which may be of microbial origin, e.g. derived from a bacterium or fungus, such as a strain of Aspergillus, in particular of A. aculeatus, A. niger, A. awamori, or A. tubigensis, from a strain of Trichoderma, e.g. T. reesei, or from a strain of Humicola, e.g. H. insolens.

The protease may be from Bacillus, e.g. B. amyloliquefaciens.

The oxidoreductase may be a glucose oxidase, a hexose oxidase, a lipoxidase, a peroxidase, or a laccase.

### Dough and/or Bread-Improving Additive

The starch-degrading glucogenic exo-amylase of Family 13 may be provided as a dough and/or bread improving additive in the form of a granulate or agglomerated powder. The dough and/or bread improving additive preferably may particularly have a narrow particle size distribution with more than 95% (by weight) of the particles in the range from 25 to 500 μm.

Granulates and agglomerated powders may be prepared by conventional methods, e.g. by spraying the amylase onto a carrier in a fluid-bed granulator. The carrier may consist of particulate cores having a suitable particle size. The carrier may be soluble or insoluble, e.g. a salt (such as NaCl or sodium sulfate), a sugar (such as sucrose or lactose), a sugar alcohol (such as sorbitol), starch, rice, corn grits, or soy.

### Alignment and Identity

For purposes of the present invention, alignments of amino acid sequences and calculation of identity scores were done using the software Align, a Needleman-Wunsch alignment (i.e. global alignment), useful for both protein and DNA alignments. The default scoring matrices BLOSUM50 and the identity matrix are used for protein and DNA alignments respectively. The penalty for the first residue in a gap is −12 for proteins and −16 for DNA, while the penalty for additional residues in a gap is −2 for proteins and −4 for DNA. Align is from the FASTA package version v20u6 (W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA”, Methods in Enzymology, 183:63-98).

### EXAMPLES

### Preparation Example: Cloning of Thermotoga neapolitana TMG Homolog SWALL: O86959, EMBL AJ009832

### Cloning

Chromosomal DNA of T. neapolitana strain DSM 4359 was isolated by QlAmp Tissue Kit (Qiagen, Hilden, Germany). The putative glucosidase gene was amplified by PCR using T. neapolitana genomic DNA as template and two oligonucleotide primers (oth88 and oth89: SEQ ID NOS: 3 and 4). The 2 primers were designed from the known DNA sequence and a Ndel site and a Notl site were incorporated in the 5′ end of oth88 and oth89, respectively. The DNA fragment was amplified with “Expand High Fidelity PCR System” (Boehringer Mannheim, Germany) using the following conditions: 94° C. for 2 min followed by 30 cycles of; 94° C. for 15 sec. 55° C. for 30 sec. 68° C. for 2 min, and ending with one cycle at 68° C. for 10 min. The amplified fragment was digested with Ndel and Notl and inserted in the expression vector pET44a (Novagen). The nucleotide sequence of the insert in the final clone was confirmed to be identical to the known sequence.

### Expression and Purification of the Recombinant Thermotoga neapolitana Enzyme

E. coli cells (BL21 Star (DEA3)pLysS (Novagen) containing the expression construct were grown in LB media+chloramphenicol (6 ug/ml). After 2.5 h expression was induced by adding IPTG to a final conc. of 0.5 mM. The cells were harvested 4 h after induction. The cells were resuspended in PBS—buffer, PH 7.3 (137 mM NaCl, 2.7 mM KCl, 4.3 mM Na2HPO4*7H2O, 1.4 mM KH2PO4) and sonicated. Cell debris was spun down and the supernatant containing the enzyme was incubated at 80° C. for 15 min, centrifuged at 20,000 rpm for 30 min at 4° C. The supernatant contained the enzyme.

### Example 1

### Starch-Degrading Glucogenic Exo-Amylase of Family 13 from T. maritima (TMG)

Doughs were made from 1 kg of flour using the European Straight dough procedure with addition of exo-amylase from T. maritima. The dosage was 5 mg enzyme protein per kg flour. A control was made without addition of the exo-amylase.

The doughs were baked into loaves of bread. The bread was wrapped and stored up to a week at ambient temperature. Firmness of the loaves was measured as described in WO 9953769. The results were as follows:

| | Invention | Control |
|---|---|---|
| 0 day | 267 | 256 |
| 1 day | 569 | 539 |
| 4 days | 1071 | 1162 |
| 7 days | 1183 | 1582 |

Elasticity of the loaves was measured as described in U.S. Pat. No. 6,162,628. The results were as follows:

| | Invention | Control |
|---|---|---|
| 0 days | 66.3 | 66.3 |
| 1 day | 62.0 | 61.5 |
| 4 days | 55.4 | 54.2 |
| 7 days | 50.3 | 49.6 |

The results show that the glucogenic exo-amylase has anti-staling performance as it softens the crumb (reduced firmness) and slightly improves the elasticity after storage.

1-13. (canceled)

14. A method of producing a variant polypeptide, which method comprises:
a) providing an amino acid sequence and a three-dimensional model for a fungal alpha-amylase and for a maltogenic alpha-amylase wherein one or both models includes a substrate,
b) superimposing the two three-dimensional models,
c) selecting an amino acid residue in the fungal amylase which has a C-alpha atom located>0.8 Å from the C-alpha atom of any amino acid residue in the maltogenic alpha-amylase and <11 Å from an atom of a substrate,
d) altering the fungal amylase sequence wherein the alteration comprises substitution or deletion of the selected residue or by insertion of a residue adjacent to the selected residue, and
e) producing the polypeptide having the resulting amino acid sequence.

15. The method of claim 14 wherein the substitution or insertion is made with an amino acid residue of the same type as the corresponding residue in the maltogenic alpha-amylase sequence, wherein the type is positively charged, negatively charged, hydrophilic or hydrophobic.

16. The method of claim 14 wherein the substitution or insertion is made with a larger or smaller amino acid residue depending on whether the corresponding residue in the maltogenic alpha-amylase sequence is larger or smaller.

17. The method of claim 14 wherein the alteration of the amino acid sequence further comprises substitution of a fungal alpha-amylase residue which has a C-alpha atom located less than 11 Å from an atom of a substrate and<0.8 ↑ from the C-alpha atom of a maltogenic alpha-amylase residue.

18. The method of claim 17 wherein the substitution is made with an amino acid residue of the same type as the corresponding maltogenic alpha-amylase residue, wherein the type is positive, negative, hydrophilic or hydrophobic.

19. A polypeptide which:
a) has an amino acid sequence having at least 70% identity to SEQ ID NO: 2; and
b) compared to SEQ ID NO: 2 comprises an amino acid alteration which is a deletion, substitution or insertion at a position corresponding to 15, 32-36, 63-64, 73-77, 119-120, 125-126, 151-152, 155-156, 167-172, 211 or 233-239,
c) has the ability to hydrolyze starch.

20. The polypeptide of claim 19 wherein said polypeptide:
a) has an amino acid sequence having at least 80% identity to SEQ ID NO: 2; and
b) compared to SEQ ID NO: 2 comprises an amino acid alteration which is a deletion, substitution or insertion at a position corresponding to 15, 32-36, 63-64, 73-77, 119-120, 125-126, 151-152, 155-156, 167-172, 211 or 233-239,
c) has the ability to hydrolyze starch.

21. The polypeptide of claim 19 wherein said polypeptide:
a) has an amino acid sequence having at least 90% identity to SEQ ID NO: 2; and
b) compared to SEQ ID NO: 2 comprises an amino acid alteration which is a deletion, substitution or insertion at a position corresponding to 15, 32-36, 63-64, 73-77, 119-120, 125-126, 151-152, 155-156, 167-172, 211 or 233-239,
c) has the ability to hydrolyze starch.

22. A polypeptide of claim 19 wherein said polypeptide:
a) has an amino acid sequence having at least 95% identity to SEQ ID NO: 2; and
b) compared to SEQ ID NO: 2 comprises an amino acid alteration which is a deletion, substitution or insertion at a position corresponding to 15, 32-36, 63-64, 73-77, 119-120, 125-126, 151-152, 155-156, 167-172, 211 or 233-239,
c) has the ability to hydrolyze starch.

23. The polypeptide of claim 19 wherein the alteration comprises substitution or insertion with an amino acid residue of the same type as the corresponding residue in the maltogenic alpha-amylase sequence, wherein the type is positively charged, negatively charged, hydrophilic or hydrophobic.

24. The polypeptide of claim 19 wherein the alteration comprises substitution or insertion with a larger or smaller amino acid residue depending on whether the corresponding residue in the maltogenic alpha-amylase sequence is larger or smaller.

25. The polypeptide of claim 19 comprising alteration corresponding to Q35K/R, Y75A/F, Y155W, L166F, G167T, N169P, T170A, L232Y, D233G, G234D, Y252F, Y256T, 166LGDNTV171 to FTDPAGF, 168-171 (DNTV) substituted with DPAGF, 168-171 (DNTV) substituted with DPAGL, 168-171 (DNTV) substituted with DPAGC.

26. The polypeptide of claim 19 which has the amino acid sequence of SEQ ID NO: 2 comprising one or more of the following alterations:
Q35K/R
Y75A/F
Y155W
L166F
G167T
N169P
T170A
L232Y
D233G
G234D
Y252F
Y256T
166LGDNTV171 to FTDPAGF
168-171 (DNTV) substituted with DPAGF
168-171 (DNTV) substituted with DPAGL
168-171 (DNTV) substituted with DPAGC
D233G+G234D
Q35K+Y75F+D168Y
Q35R+Y75F
Q35R+Y75F+D168Y
168-171 (DNTV) substituted with DPAGF+Y75A
168-171 (DNTV) substituted with DPAGF+Q35K+Y75A
168-171 (DNTV) substituted with DPAGF+Q35K+Y75A+D233G+G234D
168-171 (DNTV) substituted with DPAGF+Y75A+G234D
168-171 (DNTV) substituted with DPAGF+Y75A+D233G+G234D
166-171 (LGDNTV) substituted with FTDPAGF+Y75A
166-171 (LGDNTV) substituted with FTDPAGF+Q35K+Y75A
166-171 (LGDNTV) substituted with FTDPAGF+Q35K+Y75A+D233G+G234D

27. A polypeptide which:
a) has an amino acid sequence having at least 70% identity to SEQ ID NO: 3;
b) compared to SEQ ID NO: 3 comprises an amino acid alteration which comprises Q35K, Q35R, P70K, L151F, L151D, N233G+G234D, D75G, D75A or 166-171 (EGDTIV) substituted with FTDPAGF, and
c) has the ability to hydrolyze starch.

28. The polypeptide of claim 27 wherein said polypeptide:
a) has an amino acid sequence having at least 80% identity to SEQ ID NO: 3;
b) compared to SEQ ID NO: 3 comprises an amino acid alteration which comprises Q35K, Q35R, P70K, L151F, L151D, N233G+G234D, D75G, D75A or 166-171 (EGDTIV) substituted with FTDPAGF, and
c) has the ability to hydrolyze starch.

29. The polypeptide of claim 27 wherein said polypeptide:
a) has an amino acid sequence having at least 90% identity to SEQ ID NO: 3;
b) compared to SEQ ID NO: 3 comprises an amino acid alteration which comprises Q35K, Q35R, P70K, L151F, L151D, N233G+G234D, D75G, D75A or 166-171 (EGDTIV) substituted with FTDPAGF, and
c) has the ability to hydrolyze starch.

30. The polypeptide of claim 27 wherein said polypeptide:
a) has an amino acid sequence having at least 95% identity to SEQ ID NO: 3;
b) compared to SEQ ID NO: 3 comprises an amino acid alteration which comprises Q35K, Q35R, P70K, L151F, L151D, N233G+G234D, D75G, D75A or 166-171 (EGDTIV) substituted with FTDPAGF, and
c) has the ability to hydrolyze starch.

31. A polypeptide which:
a) has an amino acid sequence having at least 70% identity to SEQ ID NO: 4;
b) compared to SEQ ID NO: 4 comprises an amino acid alteration which comprises G35K, G35R, A76del+D77del, D74del+A78del, D74A, D74G, D77A, D77G, Y157W or L168F+A169T+T171P+P172A+T173G, and
c) has the ability to hydrolyze starch.

32. The polypeptide of claim 31 wherein said polypeptide:
a) has an amino acid sequence having at least 80% identity to SEQ ID NO: 4;
b) compared to SEQ ID NO: 4 comprises an amino acid alteration which comprises G35K, G35R, A76del+D77del, D74del+A78del, D74A, D74G, D77A, D77G, Y157W or L168F+A169T+T171P+P172A+T173G, and
c) has the ability to hydrolyze starch.

33. The polypeptide of claim 31 wherein said polypeptide:
a) has an amino acid sequence having at least 90% identity to SEQ ID NO: 4;
b) compared to SEQ ID NO: 4 comprises an amino acid alteration which comprises G35K, G35R, A76del+D77del, D74del+A78del, D74A, D74G, D77A, D77G, Y157W or L168F+A169T+T171P+P172A+T173G, and
c) has the ability to hydrolyze starch.

34. The polypeptide of claim 31 wherein said polypeptide:
a) has an amino acid sequence having at least 95% identity to SEQ ID NO: 4;
b) compared to SEQ ID NO: 4 comprises an amino acid alteration which comprises G35K, G35R, A76del+D77del, D74del+A78del, D74A, D74G, D77A, D77G, Y157W or L168F+A169T+T171P+P172A+T173G, and
c) has the ability to hydrolyze starch.

35. A process for preparing a dough or a baked from dough product which comprises adding the polypeptide of claim 19.
